(19) United States
(12) Patent Application Publication
(10) Pub. No.: US 2026/0007851 A1
(43) Pub. Date: Jan. 8, 2026

(54) ELECTRICAL STIMULATION DEVICE AND ELECTRICAL STIMULATION METHOD USING SAME

(71) Applicant: KT&G CORPORATION, Daejeon (KR)

(72) Inventors: Won Kyeong Lee, Namyangju-si (KR); Sung Wook Yoon, Suwon-si (KR); Min Ju Han, Seoul (KR)

(73) Assignee: KT&G CORPORATION, Daejeon (KR)

(21) Appl. No.: 19/259,677

(22) Filed: Jul. 3, 2025

(30) Foreign Application Priority Data
Jul. 5, 2024 (KR) 10-2024-0088780
Jul. 8, 2024 (KR) 10-2024-0089540

(51) Int. Cl. A61M 21/00; A61N 1/04; A61N 1/36; G06F 3/01; G06T 11/00

(52) U.S. Cl. CPC A61M 21/00; A61N 1/0456; A61N 1/0472; A61N 1/36031; A61N 1/36036; G06F 3/015; G06T 11/00; A61M 2021/005; A61M 2021/0072

(57) ABSTRACT

An electrical stimulation device and an electrical stimulation method using the same are disclosed. The electrical stimulation method according to various embodiments of the present invention is an electrical stimulation method using an electrical stimulation device for for applying an electrical stimulation to a user's body, wherein the electrical stimulation device includes at least one electrode unit, and the method includes: a step of applying an electrical signal percutaneously through the electrode unit to at least a part of an auricular vagus nerve region of the user; and a step of collecting biometric information of the user generated by applying the electrical signal.

### CROSS-REFERENCE TO RELATED APPLICATION

This application claims priority to Korean Patent Application No. 10-2024-0088780 filed on Jul. 5, 2024, and Korean Patent Application No. 10-2024-0089540 filed on Jul. 8, 2024, the disclosure of which is incorporated herein by reference in its entirety.

### FIELD OF TECHNOLOGY

Various embodiments of the present invention relate to a device and method that enable more efficient electrical stimulation of a user's body.

### BACKGROUND

Conventional brain stimulation methods proceed mainly through direct brain stimulation of the frontal lobe, the occipital lobe, and the like. This existing brain stimulation method is a method in which a pad including an electrode connected to an electrical stimulation device is attached to the forehead or the like to perform stimulation on the brain based on a profile of a pulse interval and pulse lengths, thereby changing the shape of a user's brain wave to prove a desired effect.

There is a need to provide a scalable stimulation mode in which a better brain stimulation effect may be generated in the brain stimulation method of attaching to the forehead or the like.

### SUMMARY

The technical problem to be achieved by the present invention has been devised in order to solve the problems described above, and an object of the present invention is to provide a device and method for expanding a brain stimulation region from an existing forehead region to an ear region.

In addition, embodiments of the present invention provide a device and method for collecting a smoking event from an external device to provide a virtual smoking effect and provide a function such as monitoring a biological state.

The problems to be solved by the present invention are not limited to the problems mentioned above, and other problems not mentioned will be clearly understood by a person skilled in the art from the following description.

According to one or more example implementations of the present disclosure, an electrical stimulation method according to an embodiment of the present invention is an electrical stimulation method using an electrical stimulation device for applying an electrical stimulation to a user's body, wherein the electrical stimulation device includes at least one electrode unit, and the method includes: a step of applying an electrical signal percutaneously through the electrode unit to at least a part of anauricular vagus nerve region of the user; and a step of collecting biometric information of the user generated by applying the electrical signal.

An electrical stimulation device according to an embodiment of the present invention may include: a body that may be attached to or detached from at least a part of a user body; and an electrode unit implemented at a specific position of the body to apply electrical stimulation to anauricular vagus nerve region of a user.

An electrical stimulation device according to an embodiment of the present invention may include: a body wearable on a user's body; a stimulation unit formed at a specific position of the body of the user; a display; and a control unit configured to apply an electrical stimulation to anauricular vagus nerve of a user, generate virtual content based on biometric information collected corresponding to the electrical stimulation, and output the virtual content through the display.

By extending the brain stimulation region from the existing forehead stimulation region to the ear region according to the embodiment of the present invention, more efficient brain/neural stimulation may be performed and user convenience may be increased.

In addition, while smoking awakening is achieved by an electrical stimulation device, smoking event information is collected from an external device, and thus virtual information is provided in an augmented reality manner, so that a user may experience an environment similar to that of actual smoking.

The effects of the present invention are not limited to those mentioned above, and other effects not mentioned will be clearly understood by those skilled in the art from the following description.

### BRIEF DESCRIPTION OF THE DRAWINGS

FIG. 1 is an illustrative diagram showing an auricular vagus nerve stimulation region according to an embodiment of the present invention.

FIG. 2 to FIG. 3 are exemplary graphs showing biological information according to electrical stimulation according to an embodiment of the present invention.

FIG. 4 is an exemplary diagram showing an electrical stimulation device according to an embodiment of the present invention.

FIGS. 5 to 6 are illustrative diagrams showing a stimulation unit of the electrical stimulation device according to various embodiments of the present invention.

FIGS. 7A-7B are exemplary diagrams of an electrode unit according to an embodiment of the present invention.

FIGS. 8A-8B are exemplary diagrams showing impedance of a conventional electrode unit and impedance according to an embodiment of the present invention.

FIG. 9 is a flowchart of an electrical stimulation method according to an embodiment of the present invention.

FIG. 10 is a conceptual diagram of the electrical stimulation device and an aerosol generation device according to an embodiment of the present invention.

FIG. 11 is an exemplary diagram of the electrical stimulation device according to an embodiment of the present invention.

FIG. 12 is a configuration diagram of the electrical stimulation device according to an embodiment of the present invention.

FIG. 13 is a flowchart illustrating an operation of the electrical stimulation device according to an embodiment of the present invention.

FIG. 14 is an exemplary view of a screen viewable through a display of the electrical stimulation device according to an embodiment of the present invention.

### DETAILED DESCRIPTION

Hereinafter, preferred embodiments of the present invention will be described in detail with reference to the accompanying drawings.

However, the technical idea of the present invention is not limited to some embodiments described, but may be implemented in various forms different from each other, and one or more of the components may be selectively combined or substituted between embodiments within the scope of the technical idea of this invention.

In addition, terms (including technical and scientific terms) used in the embodiments of the present invention may be interpreted in a meaning that may be generally understood by a person skilled in the art to which the present invention belongs, unless expressly defined and described in detail, and terms that are generally used, such as terms defined in advance, may be interpreted in consideration of the meaning in the context of the related art.

In addition, the terminology used in the embodiments of the present invention is for the purpose of describing the embodiments and is not intended to limit the present invention.

As used herein, the singular forms “a”, “an”, and “the” may include plural forms as well, unless otherwise specified in the context, and may include one or more of all combinations that may be combined into “A”, “B”, and “C” when described as “at least one (or one or more) of A and (as well as) B and C”.

In the description of the components of the embodiments of the present invention, the terms “first”, “second”, “A”, “B”, “(a)”, “(b)”, and the like may be used.

These terms are only used to distinguish the components from other components, and are not limited by the terms to the nature, sequence, or order of the components.

In addition, when a component is described as being “connected,” “coupled,” or “accesses” to another component, the component may be directly connected, coupled, or accessed to the other component, as well as being “connected”, “coupled”, or “connected” due to another component between the component and the other component.

Also, when described as being formed or disposed “up (upper)” or “down (lower)” each component, top of or under includes not only when two components are in direct contact with each other, but also when one or more other components are formed or disposed between the two components. In addition, when it is expressed as “up (upper) or down (lower)”, it may include not only an upward direction but also a downward direction with respect to one component.

Hereinafter, embodiments will be described in detail with reference to the accompanying drawings, and the same or corresponding components will be denoted by the same reference numerals regardless of the drawing symbols, and redundant description thereof will be omitted.

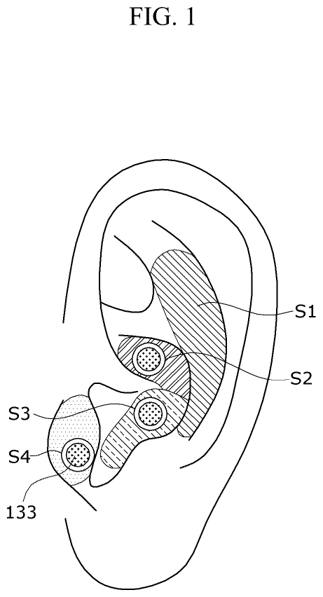

FIG. 1 is an illustrative diagram showing an auricular vagus nerve stimulation nerve stimulation region according to an embodiment of the present invention.

An auricular vagus nerve is a branch of the vagus nerve, which is a sensory nerve connected to the parasympathetic nerve, and is closely related to the regulation of the autonomic nervous system such as heart rate, digestion, and respiration. Medically, it is known to release hormones and neurotransmitters to control inflammatory responses in the body, and is also used for the management of inflammatory diseases.

As shown, the vagus nerve under the ear includes the antihelix (S1), the cymba concha (S2), the cavum concha (S3), and the tragus (S4) nerve stimulation regions. An electrode unit 133 according to various embodiments of the present invention may stimulate at least a part of theseauricular vagus nerve regions, for example, the concha-related S3, S4 regions and the tragus S4 region.

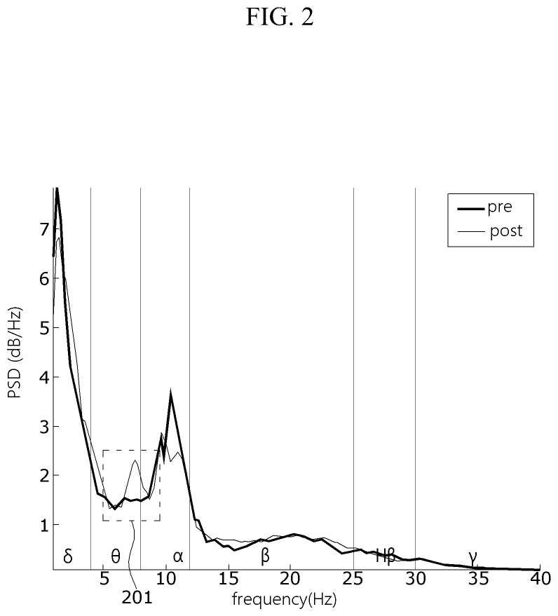

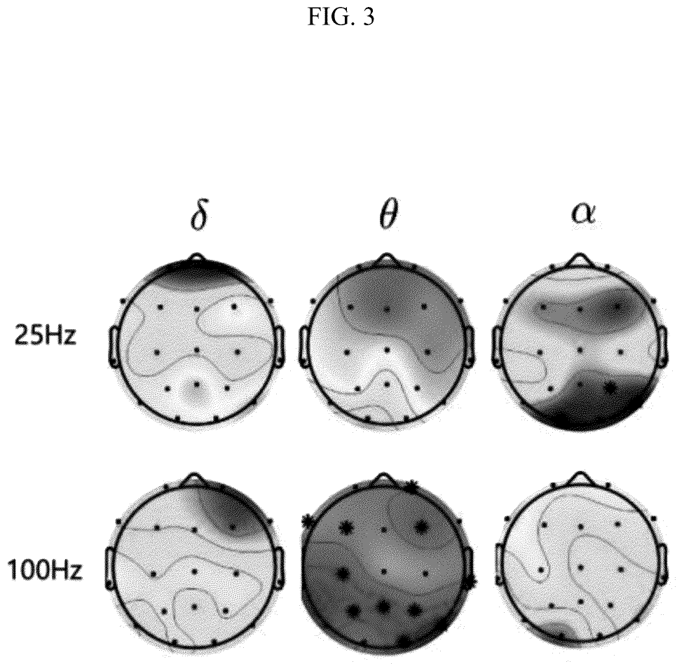

FIG. 2 to FIG. 3 are exemplary graphs showing biological information according to electrical stimulation according to an embodiment of the present invention. The band power and electroencephalogram topographical diagrams shown in FIGS. 2 to 3 were applied percutaneously to electrode units in the form of ear clips to the concha and cymba concha, and were executed under the first conditions of a frequency of 100 Hz, a pulse width of 300 μs, an inter pulse of 0 μs Biphasic, a density of 1 mA, and a duration of 15 min. In addition, as a second condition, they were executed under the following values: a frequency of 25 Hz, a pulse width of 300 μs, an inter pulse of 0 μs Biphasic, a density of 1 mA, and a duration of 15 min.

It may be experimentally confirmed that energy is increased in a theta wave region 201 as shown in FIG. 2 by applying electrical stimulation percutaneously to at least a partial region of the auricular vagus nerve.

Specifically, as a result of the average band power analysis according to the frequency band, the most noticeable increase was found in theta and low-alpha. Theta waves are cognitive/meditation waveforms that appear in the frequency domain between 4 and 8 Hz. Changes in theta waves may also be associated with a state of stability, such as sleep, or attention to processing new information, reflecting situations that require contradictory information processing and concentration control.

As the band power of theta waves increases, changes in cognitive processes such as attention, concentration, and memory appear, and an effect of improving attention concentration is also generated as norepinephrine hormone secretion is promoted.

Such an effect may provide a virtual smoking effect in an embodiment of the present invention, in particular, because it shows an effect similar to wakefulness according to a smoking effect. For example, a user wearing an electrical stimulation device 100 of the present invention may have an awakening effect such as smoking when inhaling and exhaling through an aerosol generating device 200 as electrical stimulation is applied to the auricular vagus nerve. In addition, a control unit 160 may generate virtual AR smoke content to correspond to an exhalation operation in which the user spits smoke and output the virtual AR smoke content through a display 140. Thereby, the user may experience a virtual AR smoking environment, such as actually smoking a cigarette, at the same time as feeling a smoking awakening.

FIG. 3 is biometric information showing an electroencephalogram (EEG topography) that may confirm the activation of the central part of the brain in the brain frequency band analysis. It may be confirmed through the electroencephalogram that the central part of the brain is activated by electrically stimulating the auricular vagus nerve region. Theta, measured in the central region of the brain, is related to attention, concentration, and memory.

Further, as shown in FIG. 3, a higher change may be observed experimentally when a frequency of 100 Hz is applied than when a frequency of 25 Hz is applied through the electrical stimulation device 100.

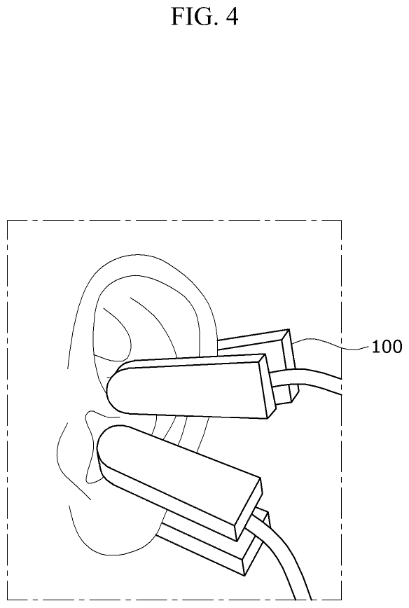

FIG. 4 is an exemplary diagram showing the electrical stimulation device 100 according to an embodiment of the present invention.

The electrical stimulation device 100 may be configured in various forms so as to be attachable or detachable from the user's body, and may generate brain stimulation by percutaneously applying the electrical stimulation to the user's auricular vagus nerve region through the electrode unit 133.

As shown in FIG. 4, the electrical stimulation device 100 may apply electrical stimulation percutaneously to the auricular vagus nerve region of the user, and collect biometric information of the user generated therefrom.

FIG. 4 shows an electrical stimulation device 100 in the form of a simple ear clip. In FIG. 4, the electrical stimulation device 100 in the form of an ear clip is a form in which an electrode unit located at one end of a clip is in contact with the vagus nerve of the ear to apply an electrical signal in a state in which the electrical stimulation device 100 is fixed to the user's ear by the clip. However, the electrical stimulation device 100 according to various embodiments of the present invention is not limited to this form.

According to various embodiments, the electrical stimulation device 100 may be implemented as a body that is removable from the user's body and at least a part of which is formed as a frame having a predetermined internal space.

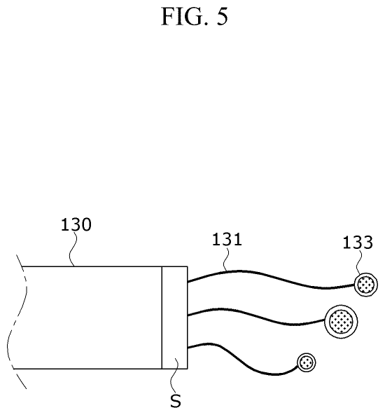

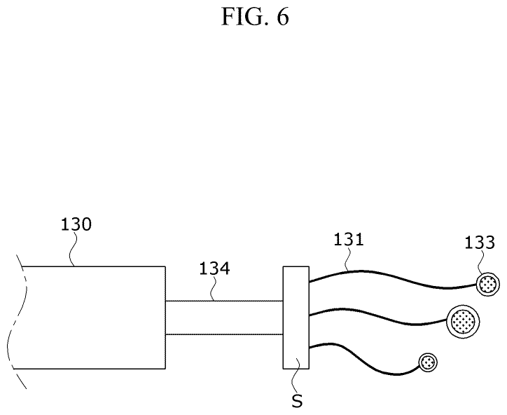

FIGS. 5 to 6 are illustrative diagrams showing a stimulation unit 130 of the electrical stimulation device 100 according to various embodiments of the present invention.

According to an embodiment, the electrical stimulation device 100 or the stimulation unit 130 may include an extension unit 131 and an electrode unit 133. An internal circuit module or a cable may be configured in the internal space of the electrical stimulation device 100 or the stimulation unit 130 so that current generated from an external power source or an internal power source is applied to the user's body through the electrode unit 133 via the extension unit 131.

The electrical stimulation device 100 may re-collect biometric information generated from the user through the internal circuit module or the cable. As an example and not by way of limitation, the cable for power supply and data communication as above may be separately configurable, but may also be implemented integrally. On the other hand, the electrical stimulation device 100 may further include a separate sensor unit to collect biometric information generated in the user's body according to electrical stimulation.

In addition, the electrical stimulation device 100 may include a control unit and a storage unit for applying an electrical stimulation and changing a frequency, and collecting and monitoring user biometric information.

The control unit may perform a data processing function of controlling power supply and signal flow between the overall operation and the internal configuration of the electrical stimulation device 100 and processing data. This control unit may include at least one processor.

The processor may execute instructions stored in a memory to generate information or control configurations of the electrical stimulation device 100. The processor may include a central processing unit (CPU), a micro processor unit (MPU), a micro controller unit (MCU), a graphic processing unit (GPU), or any other form of processor well known in the art of the present invention.

The storage unit may store data received or generated from the control unit or other components of the electrical stimulation device 100. The storage may include, for example, a memory, a cache, a buffer, or the like, and may be configured with software, firmware, hardware, or a combination of at least two or more thereof. The memory may store instructions related to the control of the electrical stimulation device 100 and data for information generation.

In FIG. 5, the extension unit 131 is shown in the form of a cable, but is not limited thereto, and may be implemented in a form having a fixed frame. In this case, the extension unit 131 may be implemented in the form of a frame bent to have a predetermined curvature so that the electrode unit 133 is introduced into the region inside the user's ear.

According to an embodiment, in FIG. 5, at least a part of the extension unit 131 may be inserted into the internal space s of the electrical stimulation device 100 or the stimulation unit 130. At least a part of the extension unit 131 or the electrode unit 133 may be introduced into the internal space s, and may be configured in such a manner that it is exposed to the outside again when an electric stimulation is to be applied.

According to various embodiments, as shown in FIG. 6, at least a partial nerve stimulation region of the electrical stimulation device 100 or the stimulation unit 130 may include a guide unit 134 so as to be adjustable in length. The guide unit 134 may be implemented in a form in which two or more nerve stimulation regions are separated from each other in a partial nerve stimulation region of the electrical stimulation device 100 or the stimulation unit 130, and may include a rail structure or the like that provides a sliding function so that a form in which the length is increased or decreased is possible. Through this guide part 134, an extra length may be provided so that the electrode unit 133 may be well fitted to the inner nerve stimulation region of the user's ear.

According to an embodiment, the guide unit 134 may be made of different materials so as to have different physical properties (e.g., strength, ductility, elasticity, etc.) from those of the extension unit 131. This makes it possible to more precisely adjust the position of the electrode unit 133.

On the other hand, apart from the above guide unit 134, each of the extension units 131 may have a different length or may be configured to be adjustable in length. In the length adjustment, for example, as the user pulls the electrode unit 133, the remaining portion of the extension unit 131 that has been in the inner space s may be additionally exposed to the outside, and at least a partial nerve stimulation region of the extension unit 131 may be pulled into the inner space, so that the length is resiliently reduced again.

As shown in FIG. 6, the physical length adjustment of the electrical stimulation device 100 or the stimulation unit 130 may be independently performed in two manners, thereby providing better convenience to the user.

On the other hand, the electrical stimulation device 100 or the stimulation unit 130 may be implemented in a flexible form in which at least some nerve stimulation regions have flexibility, may be adjusted in length, and may be bent so that electrical stimulation may be easily performed at a relatively accurate position as a specific region inside the ear. To this end, at least a part of the configurations or at least a part of physical configurations of the electrical stimulation device 100 or the stimulation unit 130 may include, but are not limited to, silicone, thermoplastic polyurethane (TPU), latex, nylon, polyurethane, thermo plastic elastomer (TPE), polyvinyl chloride (PVC), and the like.

Although not shown, according to various embodiments, the electrical stimulation device 100 may include a fixed electrical stimulation devices 100 or an insertable electrostimulating devices 100 depending on the physical form and the type of the electrode unit 133.

According to an embodiment, the fixed electrical stimulation device 100 or the stimulation unit 130 may include a frame including a ring or a hook so as to be hooked to the ear. The fixed electrical stimulation device 100 or the stimulation unit 130 may be implemented in a form in which a frame including the electrode unit 133 is branched or extended in a partial nerve stimulation region of the frame of the fixed electrical stimulation device 100 such that the electrode unit 133 may be located in an nerve stimulation region adjacent to the user's ear. The fixed electrical stimulation device 100 or the stimulation unit 130 may be formed in a structure similar to, for example, a structure of a bone conduction type earphone.

According to an embodiment, the insertable electrical stimulation device 100 or the stimulation unit 130 may be implemented in the form of a commonly used earphone, and the insertable electric stimulation device 100 or the stimulation unit 130 may include a configuration such as an earbud, an eartip, a housing, and a cable. The earbud is a part that is directly inserted into the ear and serves to transmit sound, and the eartip is attached to the end of the earbud to help it adhere to the ear. Such a eartip may be constructed from a variety of materials, such as silicone, foam, rubber, and the like. The housing is an outer case that protects the earbud and eartip, and the cable may include communication circuitry that delivers current and collects stimulation information. According to various embodiments, the earbud and the eartip may be included in the electrode unit 133 described above, and the cable may be included in the extension unit 131.

According to various embodiments, the electrode unit 133 may include at least one or more, and the electrode unit 133, as shown in FIGS. 5 to 6, may be composed of a plurality of electrode units. For example, two or three electrode units 133 may be implemented to apply an electrical stimulation to an nerve stimulation region of the auricular vagus nerve of the user.

According to various embodiments, as shown in FIGS. 5 to 6, at least a part of the plurality of electrode units 133 may have different sizes. In this way, the plurality of electrode units 133 protruding to stimulate the auricular vagus nerve are formed to have different sizes and lengths, so that each of the auricular vagus nerve regions may be more effectively stimulated.

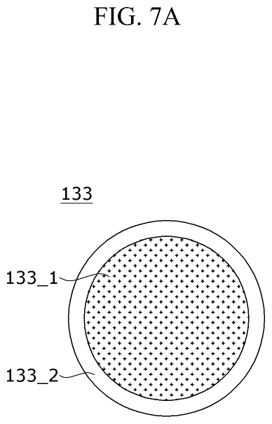

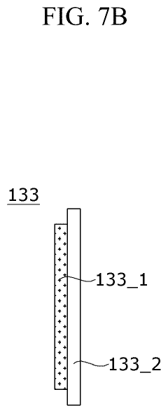

FIGS. 7A-7B are exemplary diagrams of an electrode unit 133 according to an embodiment of the present invention. According to various embodiments, the electrode unit 133 may include an electrode 133_1 and a conductive foam sheet 133_2.

The electrode 133_1 performs a function of applying an electrical stimulation to the user's body or measuring a biological signal.

According to an embodiment, the electrode 133_1 may have a predetermined nerve stimulation region and thickness. Experimentally, the nerve stimulation region of the electrode 133_1 is 78.5 mm² (with a radius of 5 mm) or more. The thickness may be preferably 0.8 mm or more, but is not limited thereto. Sufficient electrical stimulation may be difficult to apply when the electrode 133_1 has a value less than this nerve stimulation region. This size and thickness may be determined experimentally, whereby electrical stimulation may be efficiently applied to users with various physical conditions.

The conductive foam sheet 133_2 may include, but is not limited to, silicone, polyurethane, hydrogel, rubber, conductive fibers, and the like so as to maintain consistent contact and comfortably adhere to the user's skin while conducting electricity. The thickness of the conductive foam sheet 133_2 may also be set in various ways.

According to an embodiment, in order to increase electrical conductivity, a conductive foam sheet 133_2 of a wet type may be used. For example, the electrode unit 133 may include, but is not limited to, a wet electrode unit, and a dry electrode unit is also possible. In the case of a wet electrode unit, the conductive foam sheet may be configured in a manner of spraying or applying a conductive polymer or solution, and may be implemented in a manner of mixing a conductive material with an electrically conductive gel (e.g., a hydrogel) in at least a part of the foam sheet.

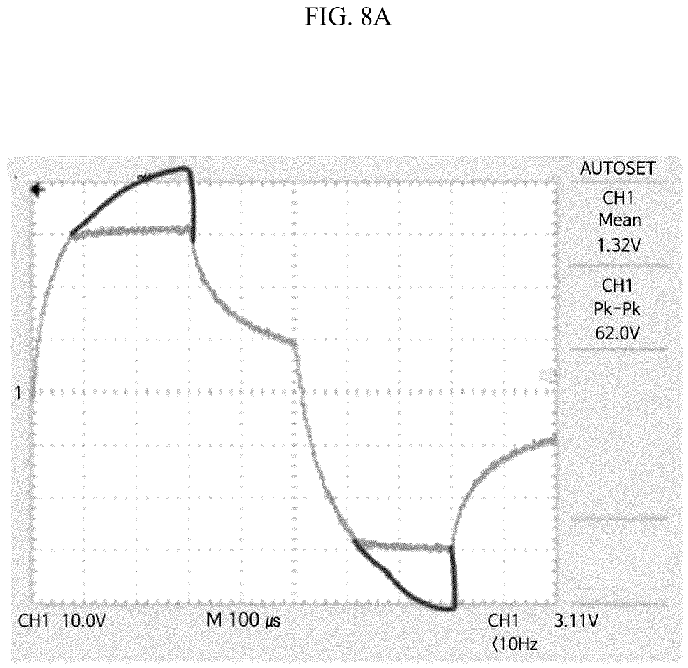

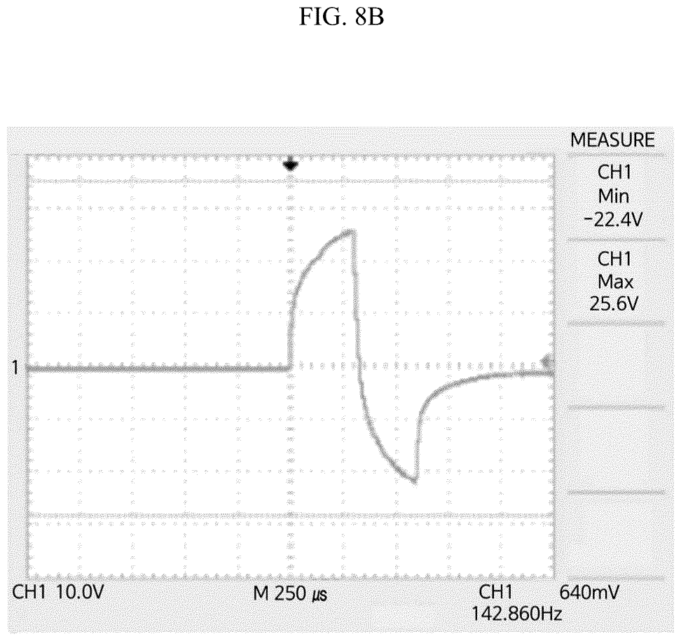

On the other hand, it is important to keep the impedance low because it may have a great influence on pain depending on its size. As shown in FIG. 8A, the impedance measured when using a conventional electrode is high due to contact with a conductive silicon electrode, so that a user may feel pain. When the configuration of the wet electrode unit or the conductive foam sheet 133_2 of the wet type is adopted, the impedance is lowered as shown in FIG. 8B, so that electrical stimulation may be performed more efficiently.

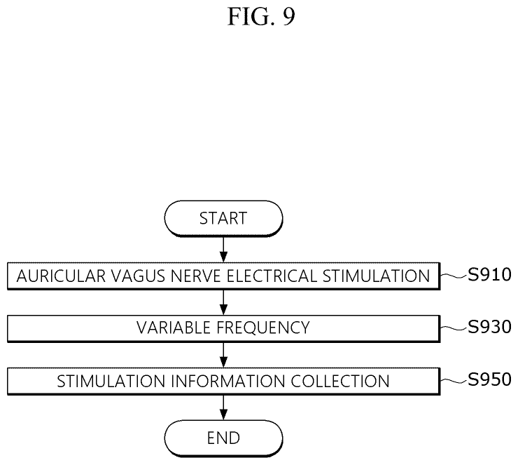

FIG. 9 is a flowchart of an electrical stimulation method according to an embodiment of the present invention. At least a part of the respective steps of FIG. 9 may be omitted or the order may be changed, and the foregoing embodiments may be performed in any step of FIG. 9.

Referring to FIG. 9, the electrical stimulation device 100 may stimulate an auricular vagus nerve of a user (S910). For example, the user may turn on the power of the electrical stimulation device (100), adjust the position of the electrode unit 133 so that the electrode unit 133 is located in the vagus nerve region inside the ear, and then apply an electric signal.

According to an embodiment, the electrical stimulation device 100 may apply an electrical signal by varying a frequency (S930). For example, the electrical stimulation device 100 may apply an electrical signal at a frequency of, for example, 20 Hz to 150 Hz. In particular, experimentally, the electrical stimulation device 100 may apply an electrical signal at a frequency of 100 HZ.

The electrical stimulation device 100 may collect stimulation information (S950). The electrical stimulation device 100 may collect biometric information of the user generated by applying the electrical signal to the auricular vagus nerve region of the user. As a result, it is possible to obtain feedback information for each user according to the auricular vagus nerve stimulation.

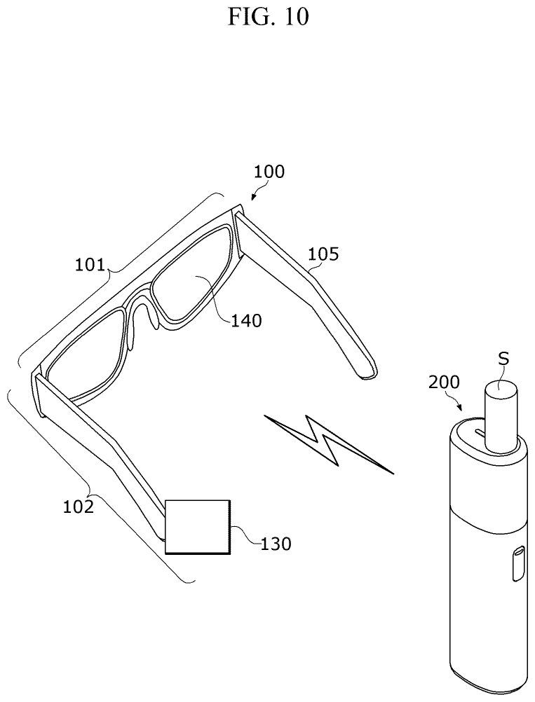

FIG. 10 is a conceptual diagram of an electrical stimulation device 100 and an external device (e.g., an aerosol generating device 200) according to an embodiment of the present invention. Such an electrical stimulation device 100 of FIG. 10 shows an embodiment configured in a different form from the electrical stimulation device 100 of FIG. 4. The electrical stimulation device 100 of FIG. 4 and at least a part of the electrical stimulation device 100 of FIG. 10 may have the same configuration (e.g., the stimulation unit 130, the electrode unit 133, and the like).

The electrical stimulation device 100 may be configured to be attachable or detachable to a user's body, and may generate brain stimulation by percutaneously applying the electrical stimulation to the auricular vagus nerve region of the user through the stimulation unit 130.

As a non-limiting embodiment, the electrical stimulation device 100 may be implemented in a wearable manner, and may apply an electrical stimulation to the auricular vagus nerve region of the user, and collect biometric information of the user generated accordingly.

In addition, the electrical stimulation device 100 may be communicatively connected to the aerosol generating device 200 to collect smoking event information generated in the aerosol generating device 200, and may output content to the display 140 together with biometric information of the user according to the electrical stimulation.

According to various embodiments, the electrical stimulation device 100 may include a body 105 that is wearable on the user's body. The body 105 forms the overall appearance of the electric stimulation device (100) and may form a frame and internal space in which components of the electric stimulation device 100 may be disposed.

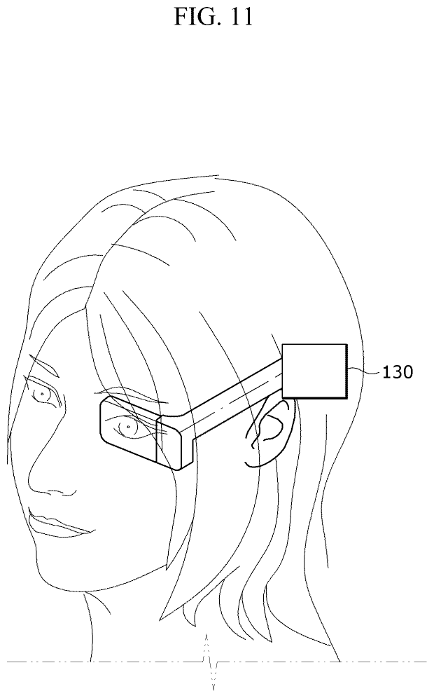

Although FIG. 10 illustrates only an embodiment in which the body 105 is formed as a smart glass in the shape of glasses as a whole, the shape of the body 105 is not limited thereto, and may also be implemented in the form of a scouting type smart glass that has one display and is formed to be hung on one ear as shown in FIG. 11.

The body 105 may include a first region 101, which is a region including at least one of the display 140, and a second region 102, which includes the stimulation unit 130.

When the electrical stimulation device 100 is configured in the form of glasses as shown in FIG. 10, the first region 101 may include a display 140 in the form of a lens in which virtual content is output in the form of augmented reality (AR) or virtual reality (VR). The first region 101 may be an nerve stimulation region representing the front (front portion) of the eyeglasses.

In addition, the second region 102 is an nerve stimulation region representing a temple (e.g., a leg portion) of the eyeglasses, and may refer to an nerve stimulation region including the stimulation unit 130 according to various embodiments of the present invention. The stimulation unit 130 may be configured to extend from in a specific nerve stimulation region of the second region 102.

The electrical stimulation device 100 may be an electronic device capable of providing an augmented reality function. The electrical stimulation device 100 may include a device configured to be worn on the user's body (e.g., a head-mounted display device (HMD) or an AR glass device in the form of glasses). Meanwhile, the electrical stimulation device 200 may be configured in a form that combines an external electronic device such as a mobile device or may communicate with an external electronic device.

The electrical stimulation device 100 may communicate with the aerosol generating device 200, which may generate a smoking event, to collect smoking event information. The aerosol generating device 200 may be, for example, an electronic cigarette in which a stick or the like having a tobacco component is inserted and heated by a heater to generate an aerosol. Although FIG. 10 illustrates an aerosol generating device having the form of an electronic cigarette in a specific form, the aerosol generating device according to various embodiments of the present invention is not limited to the form of FIG. 10.

In order to transmit smoking event information generated through the aerosol generating device 200 to the electrical stimulation device 100, at least one or a combination of the following may be included: an aerosol generating device 200 power source, a printed circuit board, a control unit (e.g., a processor), a sensor, a heater, a memory, and a communication unit. At least one of a power source, a printed circuit board, a control unit, a sensor, a heater, a memory, and a communication unit may be disposed inside the body of the aerosol generating device 200.

The user may smoke by sucking air with the upper end of the stick S exposed to the outside in his/her mouth, and smoking event information generated by smoking may be transmitted to the electrical stimulation device 100 through the communication unit of the aerosol generating device 200. To this end, the communication unit of the aerosol generating device 200 may include at least one of a mobile communication module (e.g., LTE, 4G, 5G, 6G, etc.), a wireless Internet module (e. g., Wireless LAN, Wi-Fi, etc.), and a short-range communication module (e.g., Bluetooth™, RFID, NFC, etc.).

The smoking event information is information related to a smoking event generated in the aerosol generating device 200, and may include, for example, information on whether a user is currently smoking, a smoking progress rate, a type of a cartridge or a stick, and the like.

On the other hand, the aerosol generating device 200 of FIG. 10 may also be included as the system of the present invention. For example, a brain stimulation system according to an embodiment of the present invention may include an electrical stimulation device 100 and an aerosol generating device 200.

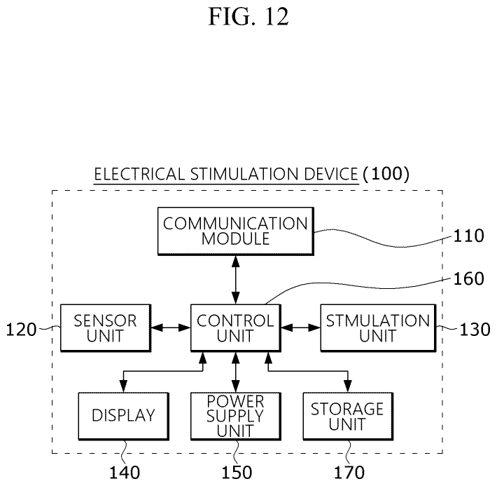

FIG. 12 is a configuration diagram of an electrical stimulation device 100 according to an embodiment of the present invention.

The electrical stimulation device 100 may include at least one of a communication module 110, a sensor unit 120, a stimulation unit 130, a display 140, a power supply unit 150, a control unit 160, and a storage unit 170, at least a part of which may be located in an internal space or a specific nerve stimulation region in the body 105 of the electrical stimulation device 100.

The communication module 110 communicates between the electrical stimulation device 100 and the aerosol generating device 200. The communication module 110 may include at least one of a mobile communication module, a wireless Internet module, and a short-range communication module.

According to an embodiment, the mobile communication module may communicate through a mobile communication network constructed according to technical standards or a communication scheme (e.g., LTE, 4G, 5G, 6G) for mobile communication.

According to an embodiment, the wireless Internet module is a module for wireless Internet connection, and may communicate through a wireless LAN (WLAN), a wireless-fidelity (Wi-Fi), a wireless fidelity (Wii-Fi) Direct, or the like.

According to an embodiment, the short-range communication module may support short-range communication by using at least one of Bluetooth™, radio frequency identification (RFID), infrared data association (IrDA), ultra wideband (UWB), ZigBee, and near field communication (NFC) technologies.

The sensor unit 120 may collect the operating state (e.g., power, temperature, etc.) of the electrical stimulation device 100 and the user's biometric information, and transmit the collected information to the control unit 160. The sensor unit 120 may include, for example, at least one of a gesture sensor, a gyro sensor, an air pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The stimulation unit 130 may be configured in various forms in at least some nerve stimulation regions (e.g., legs) of the body 105 of the electrical stimulation device 100, and may stimulate the brain by applying an electrical signal inside the user's ear. In a non-limiting embodiment, the stimulation unit 130 may also apply electrical stimulation to the brain by directly applying electrical stimulation to the forehead or head as well as theauricular vagus nerve nerve nerve stimulation region.

According to an embodiment, in the case of a device in the form of glasses having two second nerve stimulation regions, i.e., two legs, as shown in FIG. 10, the stimulation unit 130 may be formed in only one of the two second nerve stimulation regions, that is, one leg. For example, the stimulation unit 130 may be formed only in the left leg portion with respect to the front surface of the wearing user. This is because the second nerve stimulation region on the right may be connected to the heart.

The stimulation unit 130 may apply an electrical signal provided from the power supply unit 150 to a specific region of the user's body, such as a region inside the ear. The stimulation unit 130 may be formed as a frame extending or branching from at least a part of the second region 102 of FIG. 10 and having a predetermined internal space, and a cable or a circuit board for power supply and data communication may be provided in the internal space.

Although not shown, an internal circuit module or cable may be configured in the internal space of the electrical stimulation device 100 so that a current generated from the power supply 150 is applied to the user's body through the electrode unit 133 through the extension unit 131 of the stimulation unit 130. In addition, the stimulation unit 130 may re-collect biometric information generated from the user through such an internal circuit module or cable. As an example and not by way of limitation, the cable for power supply and data communication as above may be separately configurable, but may also be implemented integrally. The collected biometric information may be transferred to the control unit 160 or the storage unit 170 of the electrical stimulation device 100.

On the other hand, the electrical stimulation device 100 may collect biometric information generated in the user's body according to the electrical stimulation through the sensor unit 120 separately from the stimulation unit 130.

In the electric stimulation device 100 in the form of smart glasses, as a position in which it is easy to stimulate the vagus nerve region inside the ear, the stimulation unit 130 may be extended at one end (e.g., a leg nerve stimulation region portion) of the second region 102 of the body 105. However, the present invention is not limited thereto, and the stimulation unit 130 may be implemented in a form having a frame branched from a specific nerve stimulation region of the second region 102 and extended to an inner region of the ear.

At least some configurations of the stimulation unit 130 may be inserted into the inner space s. That is, for apparent finishing, an internal space s may be formed in at least some nerve stimulation regions on the second region 102 of the body 105 or at least some nerve stimulation regions of the region of the stimulation unit 130 extended from the at least some nerve stimulation regions. At least a part of the extension unit 131 or the electrode unit 133 may be drawn into the internal space s, and may be configured in such a manner that it is exposed to the outside again when an electric stimulation is to be applied.

Although not shown, according to various embodiments, the stimulation unit 130 may include a fixed stimulation unit or an insertable (or in-ear type) stimulation unit depending on the physical form and the type of the electrode unit 133.

According to one embodiment, the fixed stimulation unit 130 may include a frame that includes a ring or hook to be hung on the ear. The fixed stimulation unit 130 may be implemented in a form in which the frame is branched and extended at a specific point in the second region 102 of the body 105, and a frame including the electrode unit 133 may be branched or extended in a partial region of the frame such that the electrode unit 133 is located in an nerve stimulation region adjacent to the user's ear. The fixed stimulation unit 130 may be formed in a structure similar to a bone conduction type earphone, for example.

According to an embodiment, the insertable stimulation unit 130 may be implemented in the form of a commonly used earphone, and the insertable stimulation unit 130 may include a configuration such as an earbud, an eartip, a housing, and a cable. The earbud is a part that is directly inserted into the ear and serves to transmit sound, and the eartip is attached to the end of the earbud to help it adhere to the ear. Such an eartip may be constructed from a variety of materials, such as silicone, foam, rubber, and the like. The housing is an outer case that protects the earbuds and eartips, and the cable may include communication circuitry that delivers current and collects stimulation information. According to various embodiments, the earbud and the eartip may be included in the electrode unit 133 described above, and the cable may be included in an extension unit 131.

The stimulation unit 130 may include an electrode unit 133. The electrode unit 133 may include an electrode 133_1 and a conductive foam sheet 133_2.

The display 140 may visually display at least one of the electrical stimulation device 100, user information including biometric information of the user, or smoking event information. According to an embodiment, the display 140 may include a touch sensor configured to sense a touch, or a pressure sensor configured to measure an intensity of a force generated by the touch. The display 140 may be configured as an AR or VR based display that may be configured in the form of a transparent lens to provide a field of view to a user and output content at the same time.

The power supply 150 receives power from the outside of the electrical stimulation device 100 or includes an internal battery to provide a power source necessary for generating an internal circuit and a stimulation signal.

The control unit 160 may perform a data processing function of controlling power supply and signal flow between the overall operation and the internal configuration of the electrical stimulation device 100 and processing data. The control unit 160 may include at least one processor.

The processor may execute instructions stored in a memory of the storage unit 170 to generate information or control configurations of the electrical stimulation device 100. The processor may be configured to include a central processing unit (CPU), a micro processor unit (MPU), a micro controller unit (MCU), a graphic processing unit (GPU), or any other form of processor well known in the art of the present invention.

The storage unit 170 may store data received or generated from the control unit 160, the electrical stimulation device 100, or other components of the brain stimulation system. The storage unit 170 may include, for example, a memory, a cache, a buffer, or the like, and may be configured with software, firmware, hardware, or a combination of at least two or more thereof. The memory may store instructions related to the control of the electrical stimulation device 100 and data for information generation.

Although not shown in FIG. 12, the electrical stimulation device 100 may further include a camera module, an audio module for sound output, a haptic module for vibration output, and the like in order to more efficiently output content.

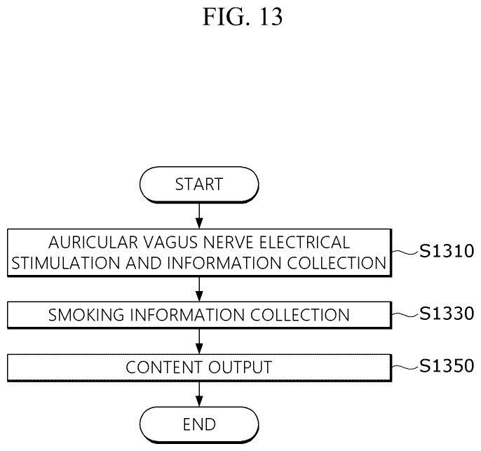

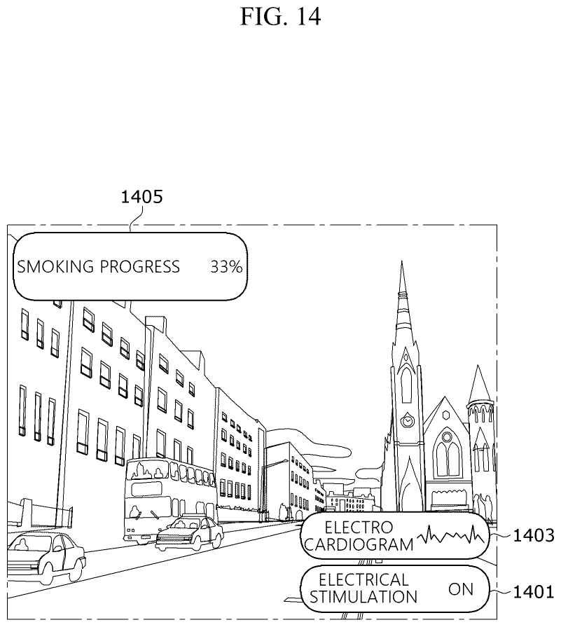

FIG. 13 is a flowchart illustrating an operation of the electrical stimulation device 100 according to an embodiment of the present invention. At least a part of the respective steps of FIG. 13 may be omitted or the order may be changed, and the foregoing embodiments may be performed in any step of FIG. 13. For ease of description of FIG. 13, refer to FIG. 14. FIG. 14 is an exemplary view of a screen viewable through the display 140 of the electrical stimulation device 100 according to an embodiment of the present invention.

Referring to FIG. 13, the electrical stimulation device 100 may stimulate theauricular vagus nerve of a user and collect information (S1310).

For example, the user may turn on the power of the electrical stimulation device 100 and wear the electrical stimulation device 100 like glasses. Then, an electrical signal is applied after adjusting the position of the stimulation unit 130 so that the electrode unit 133 is located in the inner vagus nerve region.

When the electrical stimulation device 100 applies electrical stimulation, an item 901 indicating a signal that electrical stimulation has currently occurred through the electrical stimulation device 100 is shown in FIG. 14. Through this, the user may recognize that the electric stimulation is currently in progress.

In addition, the electrical stimulation device 100 may collect biometric information generated by applying electrical stimulation to the user through the stimulation unit 130 or the sensor unit 120, and may display an AR item 903 indicating the collected biometric information through the display 140 as shown in FIG. 14. In FIG. 14, it is shown as representing an electrocardiogram, but it is not limited thereto and may represent various bio-information such as heart rate and body temperature.

Next, the electrical stimulation device 100 may collect smoking event information (S1330).

For example, a user inserts a stick for an electronic cigarette into the top of the aerosol generating device 200 and turns on the power of the aerosol generating device 200 so that it may be operated. When the user carries out an inhalation and exhalation operation with the stick in his/her mouth, the aerosol generating device 200 may transmit the user's operation and smoking event information related to the electronic cigarette to the electrical stimulation device 100. In this case, recognition of an operation such as inhalation or exhalation of the user may be performed through the electrical stimulation device 100.

Next, the electrical stimulation device 100 may output content corresponding to the smoking event information (S1350).

For example, the electrical stimulation device 100 may generate an item 905 corresponding to smoking event information (e.g., smoking progress, inhalation, and exhalation operations) received from the aerosol generating device 200. To this end, the control unit 160 may recognize the duration of the operation in which the user inhales and the time of exhalation, and output virtual cigarette smoke content through the display 140. The controller 160 may output a larger amount of smoke content through the display 140 as the duration of the inhaling operation is longer.

This content output operation is not limited to step S1350, and may be continuously performed in step S1310 and step S1330 together with information collection according to electrical stimulation.

By the above operation, the user may visually recognize the virtual cigarette smoke content while inhaling and exhaling the stick of the aerosol generating device 200 while viewing the actual scenery through the transparent display 140, and thus satisfy the smoking awakening according to the electric stimulation.

An electrical stimulation method according to an embodiment of the present invention is an electrical stimulation method using an electrical stimulation device for applying electrical stimulation to a user's body, wherein the electrical stimulation device includes at least one electrode unit, and the method includes: a step of applying an electrical signal percutaneously through the electrode unit to at least a part of the auricular vagus nerve region of the user; and a step of collecting biometric information of the user generated by applying the electrical signal.

In an electrical stimulation method according to some embodiments, the step of applying an electrical signal may include applying the electrical signal to at least one of concha, cymba concha, and tragus region of the ear.

In an electrical stimulation method according to some embodiments, the step of applying an electrical signal may include a step of varying the frequency to provide electrical stimulation.

An electrical stimulation device according to an embodiment of the present invention may include: a body that may be attached to or detached from at least a part of a user body; and an electrode unit implemented at a specific position of the body to apply electrical stimulation to a region of anauricular vagus nerve of a user.

The electrical stimulation device according to some embodiments may further include a length-adjustable extension connected to the electrode unit.

In the electrical stimulation device according to some embodiments, the electrode unit may be composed of a plurality of electrode units, and at least a part of the plurality of electrode units have different sizes.

In the electrical stimulation device according to some embodiments, the electrode unit may include an electrode and a conductive foam sheet, and the conductive foam sheet is configured in a wet manner.

An electrical stimulation device according to an embodiment of the present invention may include: a body wearable on a user's body; a stimulation unit formed at a specific position of the body of the user; a display; and a control unit configured to apply an electrical stimulation to anauricular vagus nerve of a user, generate virtual content based on biometric information collected corresponding to the electrical stimulation, and output the virtual content through the display.

The electrical stimulation device according to some embodiments may be implemented in the form of smart glasses, and the body may include: a first nerve stimulation region including the display; and a second nerve stimulation region including the stimulation unit.

In the electrical stimulation device according to some embodiments, the stimulation unit may be implemented at one end of the second nerve stimulation region.

In an electrical stimulation device according to some embodiments, the stimulation unit may include: at least one electrode unit for applying electrical stimulation; and a length-adjustable extension connected to the electrode unit.

In the electrical stimulation device according to some embodiments, the electrode unit may be composed of a plurality of electrode units, and at least a part of the plurality of electrode units may have different sizes.

In the electrical stimulation device according to some embodiments, the electrode unit includes an electrode; and a conductive foam sheet, and the conductive foam sheet is configured in a wet manner.

The electrical stimulation device according to some embodiments may further include a communication module for communicating with an external device to collect smoking event information; wherein the control unit outputs virtual content corresponding to the smoking event information through the display.

In the electrical stimulation device according to some embodiments, the stimulation unit may display virtual content generated based on the biometric information and virtual content corresponding to the smoking event information so as to be distinguished from each other on a screen output through the display.

The term ‘unit’ used in this embodiment means software or hardware components such as a field-programmable gate array (FPGA) or an ASIC, and the ‘unit’ performs certain roles. However, “unit” is not limited to software or hardware. The ‘unit’ may be configured to be in an addressable storage medium and may be configured to play back one or more processors. Thus, by way of example, ‘unit’ includes components such as software components, object-oriented software components, class components and task components, processes, functions, attributes, procedures, subroutines, segments of program code, drivers, firmware, microcode, circuitry, data, database, data structures, tables, arrays, and variables. The functionality provided within the components and ‘unit’s may be combined into a smaller number of components and ‘unit’s or further separated into additional components and ‘unit’s. In addition, the components and ‘unit’s may be implemented to play back one or more CPUs in a device or secure multimedia card.

While the foregoing has been described with reference to preferred embodiments of the invention, it will be appreciated that those skilled in the art will be able to make various modifications and changes to the invention without departing from the spirit and scope of the invention as set forth in the following claims.

What is claimed is:

1. An electrical stimulation method using an electrical stimulation device for applying an electrical stimulation to a user's body, wherein the electrical stimulation device comprises at least one electrode unit, and the method comprises:
a step of applying an electrical signal percutaneously through the electrode unit to at least a part of anauricular vagus nerve region of the user; and
a step of collecting biometric information of the user generated by applying the electrical signal.

2. The method of claim 1,
wherein the step of applying an electrical signal comprises:
applying the electrical signal to at least one of concha, cymba concha, and tragus region of the ear.

3. The method of claim 1,
wherein the step of applying an electrical signal comprises:
a step of varying the frequency to provide electrical stimulation.

4. An electrical stimulation device, comprising:
a body that may be attached to or detached from at least a part of a user body; and
an electrode unit implemented at a specific position of the body to apply electrical stimulation to anauricular vagus nerve region of a user.

5. The device of claim 4,
wherein the electrical stimulation device further comprises:
a length-adjustable extension connected to the electrode unit.

6. The device of claim 4,
wherein the electrode unit is composed of a plurality of electrode units, and
at least a part of the plurality of electrode units have different sizes.

7. The device of claim 4,
wherein the electrode unit comprises an electrode and a conductive foam sheet, and
the conductive foam sheet is configured in a wet manner.

8. An electrical stimulation device, comprising:
a body wearable on a user's body;
a stimulation unit formed at a specific position of the body of the user;
a display; and
a control unit configured to apply an electrical stimulation to anauricular vagus nerve of a user, generate virtual content based on biometric information collected corresponding to the electrical stimulation, and output the virtual content through the display.

9. The device of claim 8,
wherein the electrical stimulation device is implemented in the form of smart glasses, and
the body comprises:
a first nerve stimulation region including the display; and
a second nerve stimulation region including the stimulation unit.

10. The device of claim 9,
wherein the stimulation unit is implemented at one end of the second nerve stimulation region.

11. The device of claim 8,
wherein the stimulation unit comprises:
at least one electrode unit for applying electrical stimulation; and
a length-adjustable extension connected to the electrode unit.

12. The device of claim 11,
wherein the electrode unit is composed of a plurality of electrode units, and
at least a part of the plurality of electrode units have different sizes.

13. The device of claim 11,
wherein the electrode unit comprises:
an electrode; and
a conductive foam sheet,
and the conductive foam sheet is configured in a wet manner.

14. The device of claim 8, further comprising:
a communication module for communicating with an external device to collect smoking event information;
wherein the control unit outputs virtual content corresponding to the smoking event information through the display.

15. The device of claim 8,
wherein the stimulation unit displays virtual content generated based on the biometric information and virtual content corresponding to the smoking event information so as to be distinguished from each other on a screen output through the display.
